(19) United States
(12) Patent Application Publication
(10) Pub. No.: US 2021/0259916 A1
(43) Pub. Date: Aug. 26, 2021

(54) OZONE AND CONTRAST THERAPY SYSTEM

(71) Applicant: Gary Pylant, Spring Valley, AZ (US)

(72) Inventor: Gary Pylant, Spring Valley, AZ (US)

(21) Appl. No.: 17/180,620

(22) Filed: Feb. 19, 2021

Related U.S. Application Data
(60) Provisional application No. 62/979,802, filed on Feb. 21, 2020.

(51) Int. Cl. A61H 33/14; A61H 33/06; A61F 7/00

(52) U.S. Cl. CPC A61H 33/14; A61H 33/066; A61F 7/0053; A61H 2201/107; A61H 2033/141; A61H 2033/068; A61F 2007/0063; A61F 7/0085

(57) ABSTRACT

An ozone and contrast therapy system including an enclosure defining an interior volume, a steam system having a steam dispenser for releasing steam within the interior volume, a cold shower system having a shower head for releasing cold water within the interior volume over a user, and an ozone system having a nozzle for releasing ozone into the interior volume.

### CROSS-REFERENCE TO RELATED APPLICATIONS

This application claims the benefit of U.S. Provisional Patent Application No. 62/979,802, filed 21 Feb. 2020.

### FIELD OF THE INVENTION

This invention relates to personal care devices.

More particularly, the present invention relates to body therapy systems.

### BACKGROUND OF THE INVENTION

In the field of personal therapy, heat is often used to relax tissue, cold is used to reduce inflammation, and ozone is used in therapies to activate the immune system. Heat is often applied using saunas, spa baths, steam rooms and the like. Cold is applied using cold baths, ice and the like. Ozone therapy often refers to the process of administering ozone gas into an individual's body to treat a disease or wound. It can be used to treat medical conditions by stimulating the immune system. While each have been employed individually to various effects, each is limited in the results obtained to those touched upon previously.

It would be highly advantageous, therefore, to remedy the foregoing and other deficiencies inherent in the prior art.

An object of the present invention is to provide an ozone and contrast therapy system which provides synergy between the treatments.

### SUMMARY OF THE INVENTION

Briefly, to achieve the desired objects and advantages of the instant invention provided is an ozone and contrast therapy system including an enclosure defining an interior volume, a steam system having a steam dispenser for releasing steam within the interior volume, a cold shower system having a shower head for releasing cold water within the interior volume over a user, and an ozone system having a nozzle for releasing ozone into the interior volume.

In a specific aspect, an ozone and contrast therapy system includes an enclosure defining an interior volume. The enclosure has a base and a removable cover receivable over the base to define the interior volume. The removable cover has an opening at a head end thereof for receipt of a user's face therethrough. A body support is carried within the interior volume for supporting a user's body. The body support is positioned so that the user's body is supported within the interior volume while the user's face is positioned at and extending through the opening in the cover. A steam system has a steam generator coupled to a steam dispenser carried within interior volume for releasing steam therein. A cold shower system including a cooling unit coupled between a water source and a shower head carried by the enclosure and overlying the body support. A valve is carried within the enclosure and accessible to operation by the user. The valve is movable between an open position and a closed position wherein in the open position allows cold water from the cooling unit to exit the shower head for releasing cold water within the interior volume over the user. An ozone system has an ozone generator coupled between an oxygen source and a nozzle for releasing ozone into the interior volume.

In another aspect, an ozone and contrast therapy system includes an enclosure defining an interior volume. The enclosure includes a base, sidewalls and a top defining the interior volume, and a door carried in one of the sidewalls to provide access to the interior volume. A plurality of body supports is carried within the interior volume each for supporting a user's body. A steam system has a steam generator coupled to a steam dispenser carried within interior volume for releasing steam therein. A cold shower system has a cooling unit coupled between a water source and a plurality of shower heads carried by the enclosure and at least one of the plurality of shower heads overlying each of the plurality of body supports. Also provided is a plurality of valves, each of the plurality of valves associated with one of the plurality of shower heads. The plurality of valves is carried within the enclosure and accessible to operation by the user of the associated one of the plurality of body supports. The valves are movable between an open position and a closed position wherein in the open position allowing cold water from the cooling unit to exit the associated shower head for releasing cold water within the interior volume over the user. An ozone system has an ozone generator coupled between an oxygen source and a nozzle for releasing ozone into the interior volume. A plurality of air masks is carried within the enclosure and each is coupled to an air source outside the enclosure. Each of the plurality of air masks are associated with one of the plurality of body supports.

### BRIEF DESCRIPTION OF THE DRAWINGS

The foregoing and further and more specific objects and advantages of the invention will become readily apparent to those skilled in the art from the following detailed description of a preferred embodiment thereof, taken in conjunction with the drawings in which:

FIG. 1 is a side view of a therapy system according to the present invention;

FIG. 2 is a diagrammatic top plan view of the therapy system according to the present invention;

FIG. 3 is a diagrammatic side view of the therapy system according to the present invention; and

FIG. 4 is a schematic of a multiple person treatment facility.

### DETAILED DESCRIPTION OF THE PREFERRED EMBODIMENT

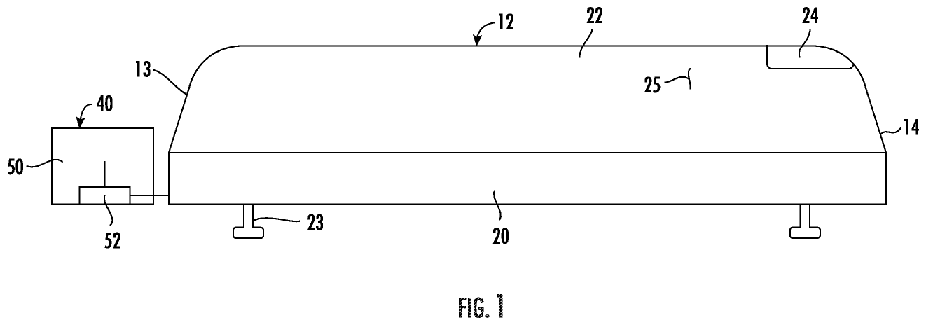

Turning now to the drawings in which like reference characters indicate corresponding elements throughout the several views, attention is directed to FIG. 1 which illustrates an ozone and contrast therapy system generally designated 10. System 10 includes an enclosure 12 having a foot end 13 and a head end 14, a steam system 15, a cold shower system 17 and an ozone system 18. Enclosure 12 includes a base 20 and a removable cover 22. Cover 22 includes an opening 24 at head end 14 for purposes which will be described presently. Base 20 is preferably supported by legs 23, but it will be understood that base 20 can be supported by a surface or other structure. Cover 22 fits to base 20 so as to define an interior volume 25 of enclosure 12 and act to constrain water, steam and ozone therein.

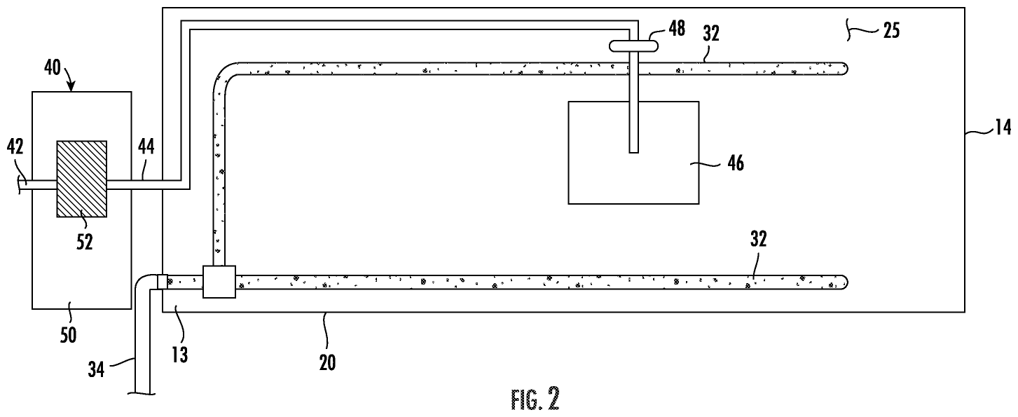

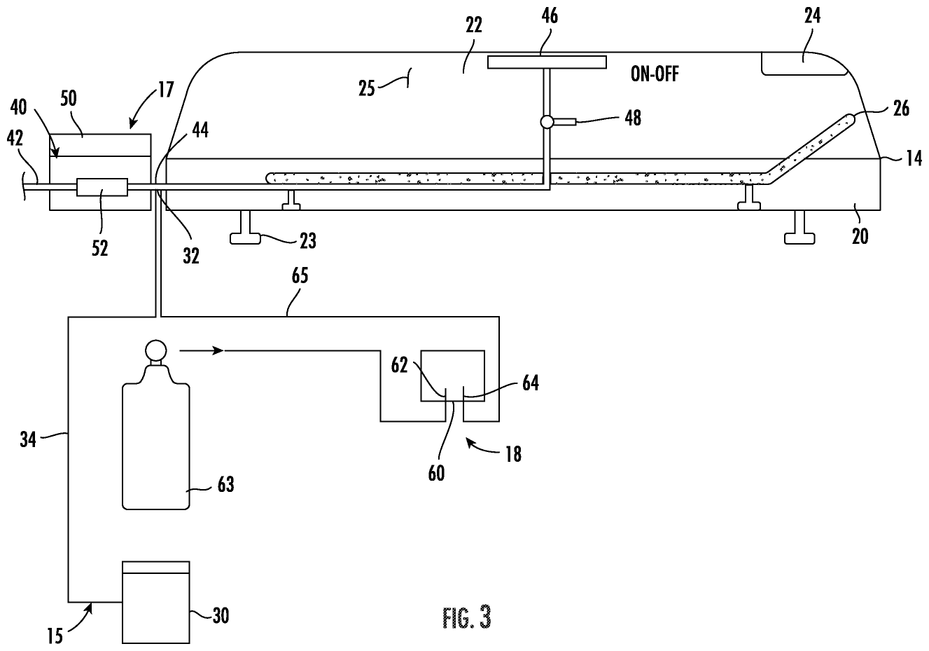

With additional reference to FIGS. 2 and 3, system 10 includes a body support 26 carried within interior volume 25 for supporting the body of a person to undergo therapy. Body support 26 supports the body of an individual above base 20 to facilitate movement of ozone, steam and cold water around the entire body below the head. Body support 26 is positioned so that the body of an individual is supported within interior volume 25 while the individual's face is positioned at and extending through opening 24. In this manner, the individual can be supplied with air from the outside atmosphere preventing breathing of ozone which is undesirable. A mask can also be fitted to an individual to ensure no ozone is inhaled.

Still referring to FIGS. 1-3, steam system 15 includes a steam generator 30 coupled to steam dispensers 32 within enclosure 12 by a conduit 34. Steam dispensers 32 preferably encircle body support 26, dispensing steam generally evenly through interior volume 25. Steam generators are well known and will not be described in further detail herein.

Cold shower system 17 includes a cooling unit 40 having an inlet 42 for receiving water from a water source, and an outlet 44. A conduit 45 extends from outlet 44 into enclosure 12 terminating in one or more shower heads 46. Shower heads 46 are positioned overlying body support 26 to facilitate dispensing of cold water on the body of an individual carried thereby. A valve or switch 48 is carried within enclosure 12, accessible to operation by an individual supported on body support 26, to supply water to the one or more shower heads 46. Valve 48 is moveable between an open position and a closed position. In the open position, cold water from cooling unit 40 passes through conduit 45 and exits one or more shower heads 46. In the closed position, water is prevented from reaching and exiting one or more shower heads 46. Cooling unit 40 can be any device for cooling water passing through. An example includes an insulted container 50 carrying a heat exchanger 52. Water from inlet 42 enters heat exchanger 52 and exits heat exchanger 52 through outlet 44. Insulated container 50 is packed with a cooling source, such as ice, which cools the water passing through heat exchanger 52. Pressure from the water source forces water through the system and out shower heads 46 when valve 48 is in the open position. A water pressure reducer can be used as well as water filters, to treat the water used.

Ozone system 18 includes an ozone generator 60 having an inlet 62 for receiving oxygen from an oxygen source 63, and an outlet 64. A conduit 65 extends from outlet 64 into enclosure 12 terminating in one or more nozzles 66. Ozone generator 60 is preferably an ozone plasma generator with a glass cavity to avoid contaminants. Ozone generated by ozone generator 60 is injected into enclosure 12 through conduit 65. Ozone permeates the atmosphere in interior void 25 completely bathing a body of an individual supported by body support 26 with ozone.

In operation, an individual reclines on body support 26 and cover 22 is positioned and coupled to base 20 with the individual's face extending from opening 24. As stated previously, an oxygen mask can be provided for the individual to insure ozone which may escape from opening 24 from being inhaled. Once closed, enclosure 12 is filled with ozone using ozone system 18. Then enclosure 12 is filled with steam from steam system 15. Steam opens pores and allows ozone to be more effective. Contrast therapy is then employed by turning valve 48 to the open position, thereby bathing the individual in ice cold water. Valve is turned to the closed position to end the cold water and continue with the steam. Thus, contrast therapy is employed by alternating the use of steam and cold water while residing in an atmosphere rich in ozone. The combination of these therapies provides a synergistic result which is greater than the combination of each alone.

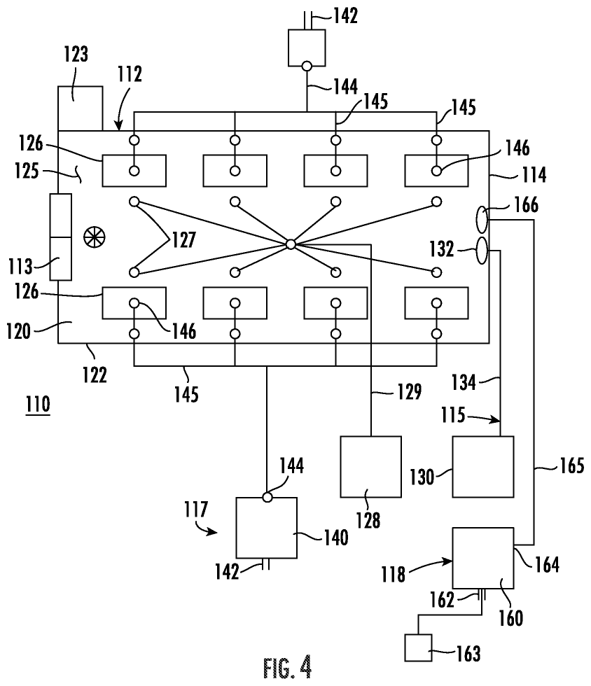

Referring now to FIG. 4, an ozone and contrast therapy system generally designated 110, which can treat multiple people at one time, is illustrated. System 110 includes an enclosure 112 suitably sized to hold numerous individuals. Enclosure 112 can be may different types of enclosures, but is preferably a mobile enclosure such as a semi-tractor trailer, cargo container and the like. Enclosure 112 incudes a door 113 at one end and an opposing end 114. Ozone and contrast therapy system 110 further includes a steam system 115, a cold shower system 117 and an ozone system 118. Enclosure 112 further includes a base 120, sidewalls 122 extending therefrom to a top 123. Enclosure 112 defines an interior volume 125 and acts to constrain water, steam and ozone therein.

Still referring to FIG. 4, system 110 includes a plurality of body supports 126 carried within interior volume 125 for supporting the body of a person to undergo therapy. Body support 126 supports the body of an individual above base 120 to facilitate movement of ozone, steam and cold water around the entire body. Body support 126 is positioned so that the body of an individual is supported within interior volume 125 and can be a reclining support (bed), sitting support (chair) and the like. Individuals to be treated within enclosure 112 each need to be fitted with an air mask 127 (such as used in scuba diving for example), allowing normal breathing and to ensure no ozone is inhaled. Breathable air is preferably provided by an air source 128 outside the enclosure and coupled to each mask 127 by a hose 129. One each of the plurality of masks is associated with one each of the plurality of body supports.

Still referring to FIG. 4, steam system 115 includes a steam generator 130 coupled to steam dispenser 132 within enclosure 112 by a conduit 134. While one steam dispenser is illustrated, it will be understood that multiple steam dispensers can be employed. Steam dispenser 132 preferably dispenses steam generally evenly through interior volume 125. Steam generators are well known and will not be described in further detail herein.

Cold shower system 117 includes a cooling unit or units 140 having an inlet 142 for receiving water from a water source, and an outlet 144. A conduit 145 extends from outlet 144 into enclosure 112 terminating in one or more shower heads 146 at each of the plurality of body supports. Shower heads 146 are positioned overlying each body support 126 to facilitate dispensing of cold water on the body of an individual carried thereby. A valve or switch is carried within enclosure 112, accessible to operation by an individual supported on one of body supports 126. Each body support 126 has an accessible valve associated therewith and with at least one shower head 146. Each valve is moveable between an open position and a closed position. In the open position, cold water from cooling unit 140 passes through conduit 145 and exits one or more shower heads 146 at the associated body support 126. In the closed position, water is prevented from reaching and exiting one or more shower heads 146. Cooling unit 140 can be any device for cooling water passing through. An example has been disclosed previously and can be used here. Access water can exit interior volume 125 through drain 147 and be collected for reuse or disposed of.

Ozone system 118 includes an ozone generator 160 having an inlet 162 for receiving oxygen from an oxygen source 163, and an outlet 164. A conduit 165 extends from outlet 164 into enclosure 112 terminating in one or more nozzles 166. Ozone generator 160 is preferably an ozone plasma generator with a glass cavity to avoid contaminants. Ozone generated by ozone generator 160 is injected into enclosure 112 through conduit 165. Ozone permeates the atmosphere in interior void 125 completely bathing a body of an individual supported by body support 126 with ozone.

In operation, system 110 can be provided at substantially any location by transporting enclosure 112 to the desired location. Once positioned, multiple individuals can be treated within enclosure 112. It will be noted that all of the generating equipment such as ozone generator 160 and steam generator 130 are located outside of interior volume 125, eliminating or reducing electromagnetic waves within enclosure 112. Individuals enter enclosure 112 through door 113 and each recline on one of body supports 126. Door 113 is closed, generally sealing interior volume 125. Each individual then dons one of oxygen masks 127 to prevent ozone within interior volume 125 from being inhaled. Once closed, enclosure 112 is filled with ozone using ozone system 118. Then enclosure 112 is filled with steam from steam system 115. Steam opens pores and allows ozone to be more effective. Contrast therapy is then employed by turning valve of the cold shower system 117 to the open position, thereby bathing individuals in ice cold water. The valve is turned to the closed position to end the cold water and continue with the steam. Thus, contrast therapy is employed by alternating the use of steam and cold water while residing in an atmosphere rich in ozone. The combination of these therapies provides a synergistic result which is greater than the combination of each alone.

Various changes and modifications to the embodiments herein chosen for purposes of illustration will readily occur to those skilled in the art. To the extent that such modifications and variations do not depart from the spirit of the invention, they are intended to be included within the scope thereof, which is assessed only by a fair interpretation of the following claims.

Having fully described the invention in such clear and concise terms as to enable those skilled in the art to understand and practice the same, the invention claimed is:

1. An ozone and contrast therapy system comprising:
an enclosure defining an interior volume;
a steam system having a steam dispenser for releasing steam within the interior volume;
a cold shower system having a shower head for releasing cold water within the interior volume over a user; and
an ozone system having a nozzle for releasing ozone into the interior volume.

2. The ozone and contrast therapy system as claimed in claim 1 wherein the enclosure further comprises:
a base; and
a removable cover receivable over the base to define the interior volume, the removable cover having an opening at a head end thereof for receipt of a user's face therethrough.

3. The ozone and contrast therapy system as claimed in claim 2 further comprising a body support carried within the interior volume for supporting a user's body, the body support being positioned so that the user's body is supported within the interior volume while the user's face is positioned at and extending through the opening in the cover.

4. The ozone and contrast therapy system as claimed in claim 1 wherein the steam system further comprises:
a steam generator; and
a conduit coupling the steam generator to the steam dispenser.

5. The ozone and contrast therapy system as claimed in claim 1 wherein the cold shower system further comprises:
a cooling unit having an inlet and an outlet;
a water source coupled to the inlet;
a conduit coupled between the cooling unit and the shower head; and
a valve carried within the enclosure and accessible to operation by the user, the valve movable between an open position and a closed position wherein in the open position allows cold water from the cooling unit to pass through the conduit and exit the shower head.

6. The ozone and contrast therapy system as claimed in claim 5 wherein the cooling unit further comprises:
an insulted container;
a heat exchanger coupled to the inlet and the outlet and carried by the insulated container; and
a cooling source carried within the insulated carrier.

7. The ozone and contrast therapy system as claimed in claim 1 wherein the ozone system further comprises:
an ozone generator having an inlet and an outlet;
an oxygen source coupled to the inlet; and
a conduit coupled between the outlet and the nozzle.

8. The ozone and contrast therapy system as claimed in claim 1 wherein the enclosure further comprises:
a base, sidewalls and a top defining the interior volume;
a door in one of the sidewalls to provide access to the interior volume; and
an air mask to be worn by a user, the air mask coupled to an air source outside the enclosure.

9. The ozone and contrast therapy system as claimed in claim 8 further comprising a plurality of body support structures carried therein, each having an associated shower head, and air mask.

10. An ozone and contrast therapy system comprising:
an enclosure defining an interior volume, the enclosure including a base and a removable cover receivable over the base to define the interior volume, the removable cover having an opening at a head end thereof for receipt of a user's face therethrough;
a body support carried within the interior volume for supporting a user's body, the body support being positioned so that the user's body is supported within the interior volume while the user's face is positioned at and extending through the opening in the cover;
a steam system including a steam generator coupled to a steam dispenser carried within interior volume for releasing steam therein;
a cold shower system including a cooling unit coupled between a water source and a shower head carried by the enclosure and overlying the body support and a valve carried within the enclosure and accessible to operation by the user, the valve movable between an open position and a closed position wherein in the open position allows cold water from the cooling unit to exit the shower head for releasing cold water within the interior volume over the user; and
an ozone system including an ozone generator coupled between an oxygen source and a nozzle for releasing ozone into the interior volume.

11. An ozone and contrast therapy system comprising:
an enclosure defining an interior volume, the enclosure including a base, sidewalls and a top defining the interior volume, a door is carried in one of the sidewalls to provide access to the interior volume;
a plurality of body supports carried within the interior volume each for supporting a user's body;
a steam system including a steam generator coupled to a steam dispenser carried within interior volume for releasing steam therein;
a cold shower system including a cooling unit coupled between a water source and a plurality of shower heads carried by the enclosure and at least one of the plurality of shower heads overlying each of the plurality of body supports, and plurality of valves, each of the plurality of valves associated with one of the plurality of shower heads, the plurality of valves carried within the enclosure and accessible to operation by the user of the associated one of the plurality of body supports, the valves movable between an open position and a closed position wherein in the open position allowing cold water from the cooling unit to exit the associated shower head for releasing cold water within the interior volume over the user;
an ozone system including an ozone generator coupled between an oxygen source and a nozzle for releasing ozone into the interior volume; and
a plurality of air masks carried within the enclosure and each coupled to an air source outside the enclosure, each of the plurality of air masks associated with one of the plurality of body supports.
